(19) United States
(12) Patent Application Publication
(10) Pub. No.: US 2026/0301937 A1
(43) Pub. Date: Oct. 1, 2026

(54) INTELLIGENT CARE DEVICE AND SYSTEM

(71) Applicant: COPLUS INC., Tainan City (TW)

(72) Inventors: Po-Hua WU, Tainan City (TW); Ming-Zhang SU, Tainan City (TW); Jia-En WU, Tainan City (TW); Yung-Chin TSAI, Tainan City (TW); Jhao-Yang HUANG, Tainan City (TW); Yueh-Cheng LIN, Tainan City (TW); Yen-Ting CHEN, Tainan City (TW); Sung-Min LIN, Tainan City (TW); Chih-Hung CHEN, Tainan City (TW)

(21) Appl. No.: 19/302,070

(22) Filed: Aug. 17, 2025

(30) Foreign Application Priority Data
Mar. 31, 2025 (TW) 114112421

(51) Int. Cl. G16H 40/67; G08B 5/36; G08B 21/04; G08B 21/14; G16H 10/60; H04W 4/80; H04W 4/90; H05K 7/20

(52) U.S. Cl. CPC G16H 40/67; G08B 5/36; G08B 21/043; G08B 21/0476; G08B 21/14; G16H 10/60; H04W 4/80; H04W 4/90; H05K 7/20145; H05K 7/2039

(57) ABSTRACT

An intelligent care device and system are provided. The intelligent care device includes a bottom portion and a top portion. The bottom portion includes: a bottom housing, a heat sink, a noise-depressing metal plate, a first fan, a motor, a first transmission shaft portion and a speaker. The top portion includes: a top housing, a second transmission shaft portion, sensors, a second computer module and a light emitting diode (LED) lamp ring. The first fan is disposed on the noise-depressing metal plate to use the noise-depressing metal to efficiently guide an air flow to the heat sink for heat dissipation. The intelligent care system includes the intelligent care device, a database system for accessing user health data, a backend manage system for user health data and a mobile electronic device. The mobile electronic device receives an alarm signal transmitted by the intelligent care device.

### CROSS-REFERENCE TO RELATED APPLICATIONS

This application claims priority to Taiwan Application Serial Number 114112421, filed Mar. 31, 2025, which is herein incorporated by reference.

### BACKGROUND

### Field of Invention

The present invention relates to an intelligent care device and an intelligent care system.

### Description of Related Art

As the population structure of society moves toward an old age composition, the topic of personnel care has received more and more attention. In order to meet the demands of personnel care, many smart care devices are developed. Smart care devices can be mainly used to determine whether the personnel are in a dangerous situation or in need for assistance, so as to achieve the purpose of personnel care.

### SUMMARY

Embodiments of the present invention provide an intelligent care device and an intelligent care system used to determine whether a person is in a dangerous environment, and accordingly sending a warning message to achieve the purpose of personnel care.

In accordance with an embodiment of the present invention, the intelligent care device includes a bottom portion and a top portion disposed on the bottom portion. The bottom portion includes: a bottom housing, two sound source positioning microphones, a first computer module, a heat sink, a noise-depressing metal plate, a first fan, a motor, a speaker, and a first transmission shaft portion. The bottom housing has a first side and a second side opposite to each other. The sound source positioning microphones are respectively disposed at the first side and the second side. The first computer module is disposed in the bottom housing and electrically connected to the sound source positioning microphones to locate a sound source in accordance with signals transmitted by the sound source positioning microphones. The heat sink is disposed on the first computer module. The noise-depressing metal plate is disposed on the heat sink and covers a side of the heat sink. The first fan is disposed on the noise-depressing metal plate, in which an air flow is guided by the noise-depressing metal to the heat sink to achieve heat dissipation. The motor is disposed in the bottom housing. The speaker is disposed in the bottom housing and electrically connected to the first computer module to send a warning message. The first transmission shaft portion is located in the bottom housing and coupled to the motor. The top portion includes: a top housing, a second transmission shaft portion, a plurality of sensors, a second computer module and a light-emitting diode (LED) lamp ring. The second transmission shaft portion is located in the top housing, in which the motor is configured to rotate the top portion though the first transmission shaft portion and the second transmission shaft portion. The sensors are disposed in the top housing. The second computer module is disposed in the top housing and electrically connected to the sensors, in which the second computer module is configured to receive and process sensed values of the sensors, and to send warning signals when one of the sensed values of the sensors is greater than a predetermined threshold value. The LED lamp ring is disposed at an inner side of the top housing, in which the LED lamp ring is configured to show a warming state or an operation state of the intelligent care device though signals.

In some embodiments, the first computer module is an artificial intelligence computing module, the artificial intelligence computing module is an embedded computer configured to perform machine vision, image recognition and sound positioning algorithms, and the first computer module has an expandable SIM card module.

In some embodiments, the top portion further includes: a camera disposed in the top housing and electrically connected to the first computer module to capture a plurality of environmental images, in which the artificial intelligence computing module determines whether an abnormal event of personnel falling or abnormal personnel moving occurs, and generates a real-time warning signal and transmits the real-time warning signal to an external electronic device when the abnormal event is detected.

In some embodiments, the intelligent care device further includes: a memory card slot and a DC jack. The memory card slot is electrically connected to the second computer module to store the environmental images captured by the camera and the sensed values of the sensors. The DC jack is configured to supply power of the intelligent care device.

In some embodiments, the second computer module includes an STM32 single chip, and the STM32 single chip is configured to receive AT commands sent from the first computer module through a UART communication interface to control the motor, the LED lamp ring and the speaker.

In some embodiments, the top portion further includes a second fan disposed in the top housing and aligned with a plurality of ventilation holes of the top housing to enhance heat dissipation performance inside the top housing and ensure the data accuracy and the data stability of the second computer module and the sensors.

In some embodiments, the sensors include a plurality of gas sensing modules to detect the concentrations of carbon monoxide, carbon dioxide and gas in the environment.

In some embodiments, the intelligent care device further includes a display panel electrically connected to the first computer module, in which the first computer module is configured to display the warning signals in red, white, and yellow emoticons on the display panel.

In some embodiments, the first computer module has a cache memory, and the cache memory is a 64-bit system, and the cache memory includes L 1, L2, and L3 caches.

In some embodiments, the second computer module has a Cortex-M core and is configured to control input or output with external devices, storage devices, external equipment and clock signals through a bus.

In some embodiments, the external devices are devices compatible with GPIO, UART, I2C, SPI, USB, ADC communication protocols.

In accordance with an embodiment of the present invention, the intelligent care system includes: the above intelligent care device, a database system for accessing user health data, a backend management system for user health data and a mobile electronic device. The database system is configured to store historical data and system configurations, and to present a chart easily understood for users on an application (App) and a web service platform. The backend management system is electrically connected to the intelligent care device and the database system, and the backend management system is configured to use a security authentication mechanism and an SQL database and adopt an MQTT protocol mechanism to be integrated with the database system for accessing user health data. The mobile electronic device is electrically connected to the intelligent care device and the backend management system to receive an alarm signal sent by the intelligent care device and to obtain the chart through the application program.

In some embodiments, the mobile electronic device is a smartphone configured to load the application to obtain the chart and display the chart to the user by using a display screen of the smartphone.

In some embodiments, the first computer module is configured to transmit the alarm signal to the smartphone through Bluetooth wireless transmission technology.

In accordance with an embodiment of the present invention, the intelligent care system includes: the above intelligent care device having the embedded computer, a database system for accessing user health data, a backend management system for user health data and a mobile electronic device. The database system is configured to store historical data and system configurations, and to present a chart easily understood for users on an application and a web service platform. The backend management system is electrically connected to the intelligent care device and the database system, and the backend management system is configured to use a security authentication mechanism and an SQL database and adopt an MQTT protocol mechanism to be integrated with the database system for accessing user health data. The mobile electronic device is electrically connected to the intelligent care device and the backend management system to receive an alarm signal sent by the intelligent care device and to obtain the chart through the application program.

In some embodiments, the mobile electronic device is a smartphone configured to load the application to obtain the chart and display the chart to the user by using a display screen of the smartphone.

In some embodiments, the first computer module is configured to transmit the alarm signal to the smartphone through Bluetooth wireless transmission technology.

### BRIEF DESCRIPTION OF THE DRAWINGS

The invention can be more fully understood by reading the following detailed description of the embodiment, with reference made to the accompanying drawings as follows:

FIG. 1 is a schematic diagram showing the appearance of an intelligent care device in accordance with an embodiment of the present invention;

FIG. 2 is a schematic diagram showing the internal structure of the intelligent care device from a first perspective in accordance with an embodiment of the present invention;

FIG. 3 is a schematic diagram showing the internal structure of the intelligent care device 100 from a second perspective in accordance with an embodiment of the present invention; and

FIG. 4 is a schematic flowchart of a puncher design method in accordance with an embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

The using of “first”, “second”, “third”, etc. in the specification should be understood for identifying units or data described by the same terminology but are not referred to particular order or sequence.

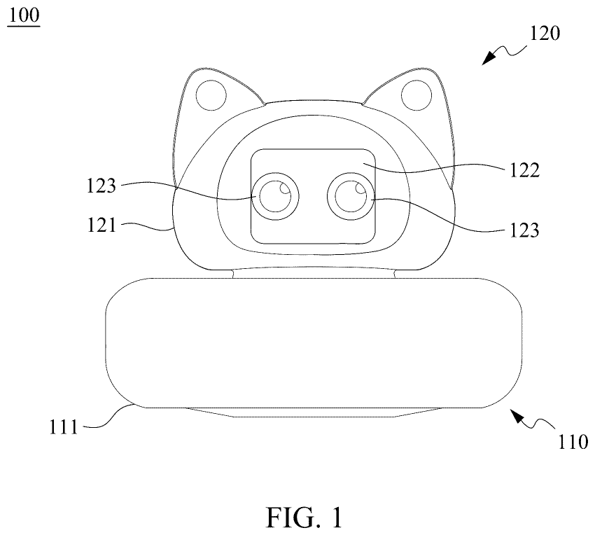

Referring to FIG. 1, FIG. 1 is a schematic diagram showing the appearance of an intelligent care device 100 in accordance with an embodiment of the present invention. The intelligent care device 100 includes a bottom portion 110 and a top portion 120. The bottom portion 110 supports the top portion 120, and plural electronic components are disposed inside a bottom housing 111 of the bottom portion 110 to drive the top portion 120 to rotate. In some embodiments, the top portion 120 can be rotated within a limited angle, but the embodiments of the present invention are not limited thereto. A display panel 122 is disposed inside a top housing 121 of the top portion 120, and can display different information to the user or make various expressions. For example, an eye structure 123 is disposed in the top portion 120, and the eye structure 123 can be used in conjunction with the display panel 122 to produce a winking expression.

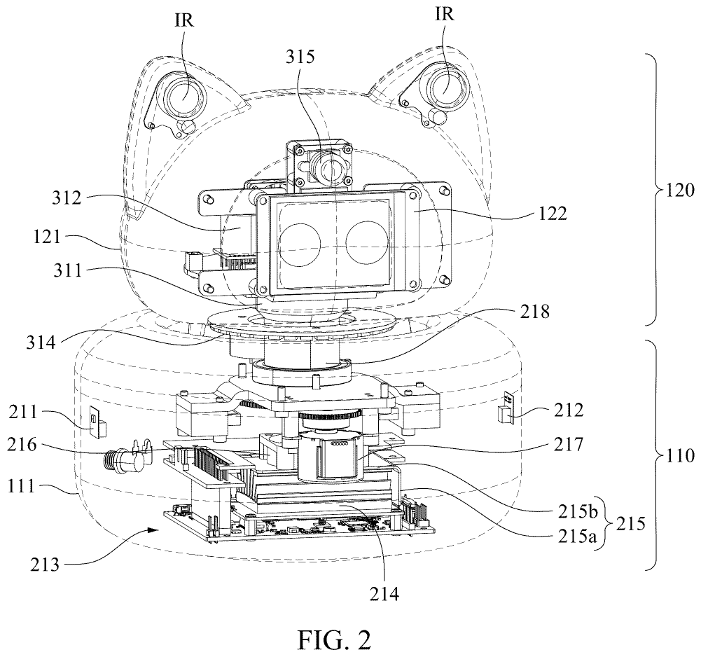

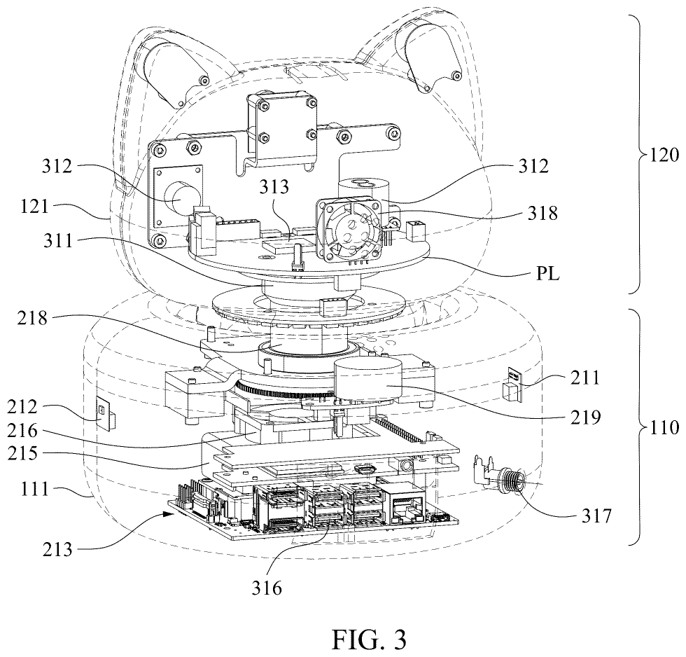

Referring to FIG. 2 and FIG. 3 simultaneously, FIG. 2 is a schematic diagram showing the internal structure of the intelligent care device 100 from a first perspective in accordance with an embodiment of the present invention, and FIG. 3 is a schematic diagram showing the internal structure of the intelligent care device 100 from a second perspective in accordance with an embodiment of the present invention. The bottom portion 110 includes: a bottom housing 111, two sound source positioning microphones 211 and 212, a first computer module 213, a heat sink 214, a noise-depressing metal plate 215, a first fan 216, a motor 217, a first transmission shaft portion 218 and a speaker 219. The bottom housing 111 has a first side and a second side opposite to each other. The sound source positioning microphones 211 and 212 are disposed in the bottom housing 111 and are located at a first side and a second side of the bottom housing 111, respectively. Since the first side and the second side of the bottom housing 111 having the largest relative distance inside intelligent care device 100, a good sound source positioning effect van be provided by the sound source positioning microphones 211 and 212.

The first computer module 213 is disposed in the bottom housing 111 and is electrically connected to the sound source positioning microphones 211 and 212. The first computer module 213 can locate a position of a sound source in accordance with the signals transmitted by the sound source positioning microphones 211 and 212. For example, when a user makes a sound at a certain position in an environment, the sound of the user is received by the sound source positioning microphones 211 and 212, and then the sound source positioning microphones 211 and 212 accordingly provide an electronic signal to the first computer module 213. The first computer module 213 can determine the user's position in accordance with the electronic signal provided by the sound source positioning microphones 211 and 212. In this embodiment, the first computer module 213 is an artificial intelligence computing module, such as an embedded computer, but the embodiments of the present invention are not limited thereto. In some embodiments, the first computer module 213 has a 64-bit system cache memory, and the cache memory includes L 1, L 2, and L3 caches. In the cache memory, memory decoding from L1 may push a cache line down into L2, and push contents into L3, and ultimately into main memory. Therefore, eviction operations from L1 are faster. When enough L2 cache is provided, the disadvantage of wasting memory by storing the content in two locations is small, and this will pay off when the eviction operations are performed, and also allow quick search for the memory card of the first computer module 213.

The heat sink 214 is disposed on the first computer module 213 to quickly guide the heat energy emitted by the first computer module 213. For example, the heat sink 214 may have fin-shaped metal plates to help the first computer module 213 to dissipate heat. The noise-depressing metal plate 215 is disposed on the heat sink 214 and covers one side of the heat sink 214. The first fan 216 is disposed above the noise-depressing metal plate 215 to provide airflow to cool the heat sink 214 and the noise-depressing metal plate 215. In this embodiment, the noise-depressing metal plate 215 is L-shaped and includes a main body portion 215a and a bent portion 215b. The main body 215a is located between the heat sink 214 and the first fan 216, and the bent portion 215b is located between the heat sink 214 and the sound source positioning microphone 212. The noise-depressing metal plate 215 can depress the noise transmitted from the heat sink 214 and reduce the interference to the electronic components in the bottom 110. For example, the noise-depressing metal plate 215 can reduce the noise interference received by the sound source positioning microphone 212. In addition, the airflow of the first fan 216 can be guided by the noise-depressing metal plate 215 to the heat sink 214 for efficient heat dissipation.

In some embodiments, the noise-depressing metal plate 215 can be designed to be U-shaped. For example, the noise-depressing metal plate 215 has a first bent portion and a second bent portion. The first bent portion may be located between the sound source positioning microphone 211 and the heat sink 214, and the second bent portion may be located between the sound source positioning microphone 212 and the heat sink 214. Therefore, the noise interference suffered by the sound source positioning microphone 211 and 212 can be further reduced. For example, the noise-depressing metal plate 215 guides the hot air flowing to the sound source positioning microphone 212 to a front side and a back side of the space of the bottom housing 111, thereby reducing the noise detected by the sound source positioning microphone 212. The noise-depressing metal plate 215 enables the cold air generated by the first fan 216 to completely pass the heat sink 214. Since the cold air is completely passing the heat sink 21 and then exhausted, no cold air escapes. Therefore, the first fan 216 can run at a lower speed, and noise resulted from heat dissipation cab be indirectly reduced.

The motor 217 is disposed in the bottom housing 111 and used to drive the top 120 to rotate. For example, the motor 217 is coupled to the first transmission shaft portion 218 to rotate the top portion 120 through the first transmission shaft portion 218 and the second transmission shaft portion 311. The first transmission shaft portion 218 is located in the bottom housing 111 and the second transmission shaft portion 311 is located in the top housing 121. In some embodiments, the motor 217 is coupled to the first transmission shaft portion 218 via gears, but the embodiments of the present invention are not limited thereto.

The speaker 219 is disposed in the bottom housing 111 and is electrically connected to the first computer module 213 for sending a warning message sent from the first computer module 213.

The top portion 120 includes: a top housing 121, a second transmission shaft portion 311, plural sensors 312, a second computer module 313 and a light emitting diode (LED) lamp ring 314. The second transmission shaft portion 311 is disposed in the top housing 121. As mentioned above, the motor 217 can rotate the top portion 120 via the first transmission shaft portion 218 and the second transmission shaft portion 311. Specifically, the second transmission shaft portion 311 is coupled to the platform PL to drive the platform PL and the entire top portion 120 to rotate. The sensors 312 are disposed in the top housing 121. In the embodiments of the present invention, the sensors 312 include plural gas sensing modules for detecting the concentrations of carbon monoxide, carbon dioxide and gas in the environment. However, the embodiments of the present invention are not limited thereto.

The gas sensing modules of the present invention may include various gas sensing components and technologies to perform accurate detection for different gases. In some embodiments, the carbon monoxide sensor may be an electrochemical sensing module, such as a ZE15-CO sensing module. The ZE15-CO sensing module has an electrochemical component inside, and generates a current signal through the electrochemical reaction between the gas, electrolytes and electrode material. The strength of the current signal is in proportional to the carbon monoxide concentration to be measured, and thus accurate concentration measurement can be performed with high selectivity and stability. In some embodiments, the carbon dioxide sensor may be a non-dispersive infrared (NDIR) sensor module, such as a MH-Z1911A sensor module. The MH-Z1911A sensor module performs gas detection by using a property that carbon dioxide gas absorbs infrared rays of specific wavelengths. When infrared rays of specific wavelengths pass through a space containing carbon dioxide gas, the energy of the infrared rays is absorbed by the gas molecules and weakened. A change of light intensity of the infrared rays can be measured by a photoelectric sensor, and the gas concentration can be estimated accordingly. The gas detection of MH-Z1911A sensor module has features of non-oxygen dependence, high selectivity and long-term stability. In some embodiments, the gas sensor may be a catalytic combustion sensing module, such as a ZS05 sensing module. A built-in sensing component of the ZS05 sensing module adopts a catalytic combustion principle. For example, a small amount of heat is generated through a catalytic oxidation reaction of gas molecules on a catalytic component on a surface of the sensing module, thereby causing temperature change of the catalytic component and resistance value change of the catalytic component. The sensor module measures the amplitude of the resistance change to estimate the gas concentration. The ZS05 sensing module has features of low power consumption, high sensitivity and good stability.

The second computer module 313 is disposed in the top housing 121 and is electrically connected to the sensor 312 for receiving and processing sensed values of the sensor 312, and configured to send warning signals when the sensed values of the sensor 312 exceed predetermined thresholds. For example, when a sensed value of the gas sensor exceeds a predetermined gas concentration threshold, the second computer module 313 sends a warning signal. For another example, when a sensed value of the carbon monoxide sensor exceeds a predetermined carbon monoxide concentration threshold, the second computer module 313 sends another warning signal. For another example, when a sensed value of the carbon dioxide sensor exceeds a predetermined carbon dioxide concentration threshold, the second computer module 313 sends another warning signal. In an embodiment of the present invention, the second computer module includes an STM32 single chip, but the embodiment of the present invention is not limited thereto.

The LED lamp ring 314 is disposed on the inner side of the top housing 121 to show a warning state or an operation state of the intelligent care device 100 through signals. The motor 217, the LED light ring 314 and the speaker 219 are controlled by the second computer module 313.

In some embodiments of the present invention, the intelligent care device 100 may further include: a camera 315, a memory card slot 316, a DC Jack 317 and a second fan 318. The camera 315 is disposed in the top housing 121 and is electrically connected to the first computer module 213. The camera 315 is configured to capture plural environmental images. The first computer module 213 (i.e., the artificial intelligence computing module) determines whether an abnormal event of personnel falling or abnormal personnel moving occurs based on the environmental images through an artificial intelligence image recognition algorithm, and generates a real-time warning signal when the abnormal event is detected, and then sends the real-time warning to a mobile electronic device.

The memory card slot 316 is disposed in the bottom housing 111 and is electrically connected to the second computer module 313 to store the environmental image captured by the camera 315 and the sensed values of the sensor 312 by a memory card. The DC jack 317 is disposed on the bottom 110 for electrically connecting to a power source (such as a mains power) to supply the power required by the smart care device 100. The second fan 318 is disposed in the top housing 121 and aligned with the plurality of ventilation holes of the top housing 121 to enhance the heat dissipation performance inside the top housing 121 and ensure the data accuracy and stability of the second computer module 313 and the sensor 312.

In some embodiments, the intelligent care device 100 may further include an infrared sensor IR electrically connected to the second computer module 313. The infrared sensor IR can emit infrared rays when the environment cannot provide sufficient light for image capture, thereby enabling the camera 315 to perform image capture though sensing the infrared rays. Therefore, the intelligent care device 100 in the embodiments of the present invention can be operated in a low-lightness environment.

It can be understood from the above descriptions that the intelligent care device 100 includes a first computer module 213 and a second computer module 313. The first computer module 213 is located at the bottom portion 110 of the intelligent care device 100 and provides an artificial intelligence computing function. The second computer module 313 is disposed on the top portion 120 of the intelligent care device 100 to receive the signals from the sensors 312 and to determine whether the surrounding environment is dangerous. The second computer module 313 can also be electrically connected to the first computer module 213 to receive AT commands sent from the first computer module 213 through a UART communication interface to control the motor 217, the LED lamp ring 314 and the speaker 219. The second computer module 313 may have a Cortex-M core, and perform input control or output control for external devices, storage devices, and external equipment through a bus by using clocks. In some embodiments, the external devices may be devices compatible with GPIO, UART, I2C, SPI, USB, or ADC communication protocols.

The operation of the intelligent care device 100 of embodiments of the present invention is described below. At first, a confirming operation is performed to determine whether a Bluetooth connection between the smart care device 100 and the external device is successful. If the connection is successful, the first computer module 213 activates the camera 315 to capture the environment images. Then, the sensed values of the sensors 312 are received through the second computer module 313. When one of the sensed values of the sensors 312 exceed a corresponding predetermined threshold, the first computer module 213 sends a warning signal to the display panel 122 to display an abnormal expression (e.g., red eyes). Therefore, the theory of color psychology can be used to present a vivid and warning image on the screen, allowing users to enhance their visual auxiliary warning response or danger association. In addition, the second computer module 313 can also be used to control the LED lamp ring 314 to emit a warning light color, such as red color. Furthermore, the second computer module 313 can also control the speaker 219 to send a warning sound.

In some embodiments, the first computer module 213 may use the environmental images to determine whether an abnormal event has occurred. For example, when the camera 315 is activated, the first computer module 213 can receive the environment image and activate an anti-theft mode. At this time, the first computer module 213 controls the display panel 122 to display an anti-theft expression. If the first computer module 213 determines that there is a stranger invades the surrounding environment of the intelligent care device 100, a warning message is issued.

In some embodiments, when the first computer module 213 determines that no abnormal event occurs, the first computer module 213 controls the display panel 122 to display normal human expressions (e.g., blinking), and the colors displayed on the display panel 122 may be blue, yellow, green, orange, and purple.

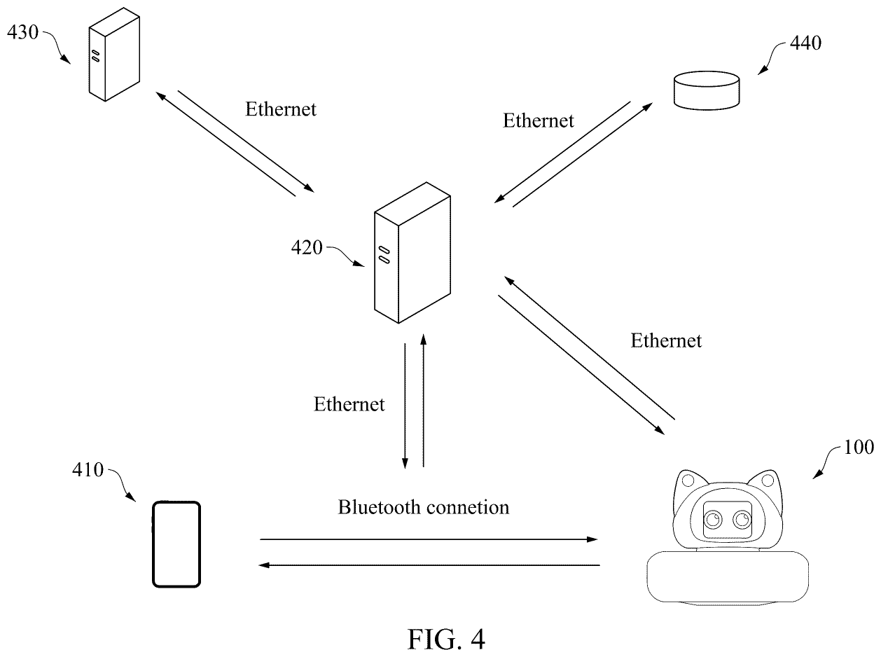

Referring to FIG. 4, FIG. 4 is a schematic diagram showing a structure of an intelligent care system in accordance with an embodiment of the present invention. The intelligent care system includes: the smart care device 100, a mobile electronic device 410, a backend management system 420 for user health data, a backend customer management web system 430, and a database system 440 for accessing user health data. The database system 440 for accessing user health data is configured to store historical data and system configurations, and to present a chart easily understood for users on an application (App) and a web service platform. The backend management system 420 is electrically connected to the smart care device 100 and the database system 440 for accessing user health data. The backend management system 420 uses a security authentication mechanism (Certificate) and an SQL database, and adopts the MQTT protocol mechanism to be integrated with the above-mentioned database system 440 for accessing user health data. The mobile electronic device 410 is electrically connected to the intelligent care device 100 and the backend management system 420 to receive an alarm signal sent by the intelligent care device 100 and to obtain the above-mentioned chart through the application. The backend customer management web system 430 is electrically connected to the backend management system 420 to perform client management with respect to the data of the backend management system 420.

Specifically, the mobile electronic device 410 can be a smartphone configured to be electrically connected to the intelligent care device 100 through Bluetooth. On the other hand, the backend management system 420 can be electrically connected to the intelligent care device 100 and the mobile electronic device 410 through Ethernet and Bluetooth. Therefore, the smartphone can load the application (App) to obtain the above-mentioned chart, and use the display screen of the smartphone to display the chart. More specifically, the smartphone may be a caregiver's smartphone. When the intelligent care device 100 transmits relevant information about the environment of the care recipient to the backend management system 420, the caregiver's mobile phone may obtain corresponding chart information to instantly understand the relevant information of the care recipient. For example, when the intelligent care device 100 determines that the gas concentration is too high, the first computer module 213 not only controls the display panel 122 to alert the care recipient, but also sends the warning signal to the caregiver's smartphone to inform the caregiver that an abnormal event has occurred.

In some embodiments, the first computer module 213 can determine whether the care recipient has fallen, and notify the caregiver an abnormal event has occurred through the warning signal. Specifically, the first computer module 213 can transmit the warning signal to the back-end management system 420 to facilitate sending a push message to the mobile phone, and to send a mail for danger warning to the mobile phone that has installed the App and has an account bound thereto.

It can be understood from the above description that the above-mentioned intelligent care system integrates the smart care device 100, the mobile electronic device 410, the backend management system 420 for user health data, the backend customer management web system 430, and the database system 440 for accessing user health data to achieve comprehensive home safety and health monitoring. The backend management system 420 is responsible for data transmission and processing, the backend customer management web system 430 provides real-time monitoring and analysis, and the database system 440 for accessing user health data ensures safe data storage and fast data access.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An intelligent care device, comprising:
a bottom portion comprising:
a bottom housing having a first side and a second side opposite to each other;
two sound source positioning microphones respectively disposed at the first side and the second side;
a first computer module disposed in the bottom housing and electrically connected to the sound source positioning microphones to locate a sound source in accordance with signals transmitted by the sound source positioning microphones;
a heat sink disposed on the first computer module;
a noise-depressing metal plate disposed on the heat sink and covering a side of the heat sink;
a first fan disposed on the noise-depressing metal plate, wherein an air flow is guided by the noise-depressing metal to the heat sink to achieve heat dissipation;
a motor disposed in the bottom housing;
a speaker disposed in the bottom housing and electrically connected to the first computer module to send a warning message; and
a first transmission shaft portion located in the bottom housing and coupled to the motor; and
a top portion disposed on the bottom portion, comprising:
a top housing;
a second transmission shaft portion located in the top housing, wherein the motor is configured to rotate the top portion though the first transmission shaft portion and the second transmission shaft portion;
a plurality of sensors disposed in the top housing;
a second computer module disposed in the top housing and electrically connected to the sensors, wherein the second computer module is configured to receive and process sensed values of the sensors, and to send warning signals when one of the sensed values of the sensors is greater than a predetermined threshold value; and
a light-emitting diode (LED) lamp ring disposed at an inner side of the top housing, wherein the LED lamp ring is configured to show a warming state or an operation state of the intelligent care device though signals.

2. The intelligent care device of claim 1, wherein the first computer module is an artificial intelligence computing module, the artificial intelligence computing module is an embedded computer configured to perform machine vision, image recognition and sound positioning algorithms, and the first computer module has an expandable SIM card module.

3. The intelligent care device of claim 2, wherein the top portion further comprises:
a camera disposed in the top housing and electrically connected to the first computer module to capture a plurality of environmental images, wherein the artificial intelligence computing module determines whether an abnormal event of personnel falling or abnormal personnel moving occurs, and generates a real-time warning signal and transmits the real-time warning signal to an external electronic device when the abnormal event is detected.

4. The intelligent care device of claim 3, further comprising:
a memory card slot electrically connected to the second computer module to store the environmental images captured by the camera and the sensed values of the sensors; and
a DC jack configured to supply power of the intelligent care device.

5. The intelligent care device of claim 1, wherein the second computer module comprises an STM32 single chip, and the STM32 single chip is configured to receive AT commands sent from the first computer module through a UART communication interface to control the motor, the LED lamp ring and the speaker.

6. The intelligent care device of claim 1, wherein the top portion further comprises:
a second fan disposed in the top housing and aligned with a plurality of ventilation holes of the top housing to enhance heat dissipation performance inside the top housing and ensure the data accuracy and the data stability of the second computer module and the sensors.

7. The intelligent care device of claim 1, wherein the sensors comprise a plurality of gas sensing modules to detect the concentrations of carbon monoxide, carbon dioxide and gas in the environment.

8. The intelligent care device of claim 1, further comprising: a display panel electrically connected to the first computer module, wherein the first computer module is configured to display the warning signals in red, white, and yellow emoticons on the display panel.

9. The intelligent care device of claim 1, wherein the first computer module has a cache memory, and the cache memory is a 64-bit system, and the cache memory includes L1, L2, and L3 caches.

10. The intelligent care device of claim 1, wherein the second computer module has a Cortex-M core and is configured to control input or output with external devices, storage devices, external equipment and clock signals through a bus.

11. The intelligent care device of claim 10, wherein the external devices are devices compatible with GPIO, UART, I2C, SPI, USB, ADC communication protocols.

12. An intelligent care system, comprising:
the intelligent care device of claim 1;
a database system for accessing user health data, wherein the database system is configured to store historical data and system configurations, and to present a chart easily understood for users on an application (App) and a web service platform;
a backend management system for user health data, wherein the backend management system is electrically connected to the intelligent care device and the database system, and the backend management system is configured to use a security authentication mechanism and an SQL database and adopt an MQTT protocol mechanism to be integrated with the database system for accessing user health data; and
a mobile electronic device electrically connected to the intelligent care device and the backend management system to receive an alarm signal sent by the intelligent care device and to obtain the chart through the application.

13. The intelligent care system of claim 12, wherein the mobile electronic device is a smartphone configured to load the application to obtain the chart and display the chart to the user by using a display screen of the smartphone.

14. The intelligent care system of claim 13, wherein the first computer module is configured to transmit the alarm signal to the smartphone through Bluetooth wireless transmission technology.

15. An intelligent care system, comprising:
the intelligent care device of claim 2;
a database system for accessing user health data, wherein the database system is configured to store historical data and system configurations, and to present a chart easily understood for users on an application (App) and a web service platform;
a backend management system for user health data, wherein the backend management system is electrically connected to the intelligent care device and the database system, and a backend management system is configured to use a security authentication mechanism and an SQL database and adopt an MQTT protocol mechanism to be integrated with the database system for accessing user health data; and
a mobile electronic device electrically connected to the intelligent care device and the backend management system to receive an alarm signal sent by the intelligent care device and obtain the chart through the application.

16. The intelligent care system of claim 15, wherein the mobile electronic device is a smartphone configured to load the application to obtain the chart and display the chart to the user by using a display screen of the smartphone.

17. The intelligent care system of claim 16, wherein the first computer module is configured to transmit the alarm signal to the smartphone through Bluetooth wireless transmission technology.
